(19) United States
(12) Patent Application Publication
(10) Pub. No.: US 2010/0120138 A1
(43) Pub. Date: May 13, 2010

(54) ENHANCED FEEDSTOCK FOR USE WITH MICRO-REFINERIES

(75) Inventor: Thomas J. Quinn, Los Gatos, CA (US)

Correspondence Address: DERGOSITS & NOAH LLP, Three Embarcadero Center, Suite 410, SAN FRANCISCO, CA, 94111, US

(73) Assignee: E-Fuel Corporation, Los Gatos, CA (US)

(21) Appl. No.: 12/688,260

(22) Filed: Jan. 15, 2010

Related U.S. Application Data
(63) Continuation-in-part of application No. 12/488,558, filed on Jul. 22, 2009.
Application No. 12/488,558 is a continuation-in-part of application No. 12/110,158, filed on Apr. 25, 2008.
Application No. 12/110,158 is a continuation-in-part of application No. 12/110,242, filed on Apr. 25, 2008.

(51) Int. Cl. C12M 1/00

(52) U.S. Cl. 435/303.1

(57) ABSTRACT

An ethanol production system includes a feedstock distributor and a micro-refinery. The feedstock distributor can pre-ferment and pre-distill the feedstock so that the ethanol content can be between 15% and 50%. This processing can improve the ethanol production efficiency because less waste material is transported from the distributor to the micro-refineries and less time is needed by the micro-refineries to produce ethanol.

### CROSS REFERENCE TO RELATED APPLICATIONS

This application is a continuation in part of U.S. patent application Ser. No. 12/488,558 which is a continuation in part of U.S. patent application Ser. Nos. 12/110,242 and 12/110,158. U.S. patent application Ser. Nos. 12/488,558, 12/110,242 and 12/110,158 are hereby incorporated by reference.

### BACKGROUND

Ethanol refineries convert feedstock into ethanol. The feedstock is fermented and the ethanol is extracted through a distillation process.

### SUMMARY OF THE INVENTION

The present invention is directed towards a system for supplying enhanced feedstock to a micro-refinery. The feedstock is fermented by the micro-refinery in a fermentation tank. Typically feedstock after that's been fermented to ethanol contains up to 15% ethanol before the yeast are killed stopping the fermentation process at around 15% ethanol. Fermentation is not a rapid process and can take several days to complete. In order to reduce this time before fermentation is complete, the feedstock distributors can provide pre-fermented feedstock that contains up to 15% ethanol. This pre-fermented feedstock can be placed in the micro-refinery and the operator can immediately begin distillation process. This is advantageous because the operator does not have to wait for their feedstock to ferment and can start producing ethanol immediately. The higher efficiency of the micro-refineries also allows the feedstock distributor to sell more feedstock because each of the micro-refineries will require more feedstock. Because the feedstock has been pre-fermented, the micro-refinery can immediately distill the feedstock using the distillation column to separate water, particulates to achieve pure ethanol.

It is also possible to further increase the ethanol content of the feedstock. To increase the ethanol output even further, the feedstock distributor can also partially distill the fermented feedstock. In an embodiment, the partial distillation can get up to 100 proof (50% ethanol) or higher before it is placed in the micro-refinery. Because the feedstock has been pre-distilled, the micro-refinery will only separate the ethanol. The distillation of pure ethanol will substantially increase with 50% ethanol feedstock versus 15%.

There are various advantages to feedstock suppliers providing pre-fermented or pre-distilled feedstock. The feedstock transportation costs are greatly improved. Normally, an operator will purchase feedstock from a distributor and the feedstock must be transported from the distributor to the micro-refinery. If unfermented feedstock is purchased, only a small percentage of this material is converted into ethanol. Even with pre-fermented feedstock only 15% of the feedstock is converted into ethanol while the remaining 85% is waste material which is mostly water. Thus, most of the feedstock that is transported is not converted into ethanol. Thus, the majority of the transportation costs are for waste material. In contrast, 50% of the 100 proof feedstock will be converted into ethanol. Thus, much more of the transported material will actually be used. This can result in a significant transportation savings since much less of the feedstock is water.

While it is possible to distill the feedstock at the distributor up to 200 proof, this may not be an efficient use of energy or distillation equipment. The energy required to distill the feedstock is not linear. Thus, the initial distillation is much more efficient than the final distillation. For example, approximately one third of the total distillation energy is used to distill from 30 proof to 100 proof. However, in order to further distill from 100 proof to 200 proof requires approximately two thirds of the total distillation energy. It can also be more efficient for the distillation to 100 proof in a large distillation system at the distributor's facilities than at the micro-refineries. However, the distillation from 100 proof to 200 proof can be equally efficient at the large distillation facility and the micro-refineries. Thus, efficiency is enhanced by this two step distillation process.

Yet another benefit of the distributor distilling the feedstock to 100 proof rather than 200 proof is improved safety. The 200 proof is substantially pure ethanol which is highly flammable. In contrast, 100 proof feedstock is half ethanol and half water. The high water content makes the 100 proof feedstock much less flammable and much safer to transport. In the event of a roadway transportation vehicle accident that results in spillage, 200 proof feedstock can easily start a fire, while the 100 proof feedstock is unlikely to ignite.

### BRIEF DESCRIPTION OF THE DRAWINGS

FIG. 1 is a diagram of a feedstock distributor and a micro refinery;

FIG. 2 is a diagram of a feedstock distributor and a micro refinery; and

FIG. 3 is a diagram of an embodiment of a micro refinery.

### DETAILED DESCRIPTION

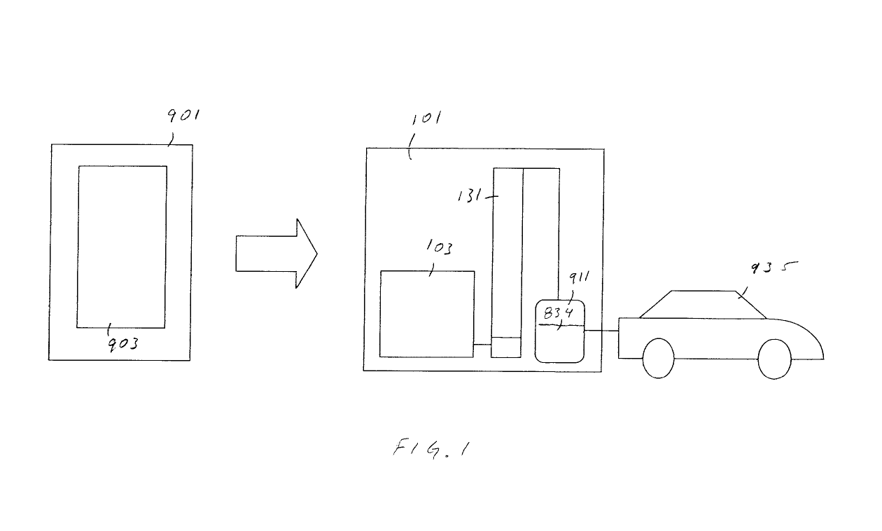

The present invention is directed towards a system for producing and distributing enhanced feedstock for micro-refineries that produce ethanol. With reference to FIG. 1, a distributor 901 provides feedstock which is transported to a micro-refinery 101. The feedstock can be mixed with water, yeast, sugar and other components in a fermentation tank 103 of the micro-refinery. Fluid mixture can ferment for several days in a fermentation tank 103. After fermentation, the liquid is distilled in a distillation tube 131 and filters to separate the ethanol from water and other contaminates. The ethanol 834 can be stored in a storage tank 911 before being pumped into an internal combustion vehicle 935. This process can be time consuming because of time delays due to the fermentation process.

In another embodiment, the distributor 901 can have a fermentation tank 903 that pre-ferments the feedstock to 30 proof. The pre-fermented feedstock can be placed in the micro-refinery 101 and immediately distilled to separate the 15% ethanol from the water in the distillation tube 131 and filtration. This can also be more efficient for the distributor and the micro-refinery operator. Rather than waiting for customers to buy feedstock, the distributor can use time to ferment the feedstock which can be sold at a higher price than unfermented feed stock. The micro-refinery operator also benefits from this, because the speed of the ethanol production is greatly enhanced since the micro-refinery 101 does not have to ferment the feedstock. In an embodiment, the pre-fermented feedstock can be between 12% to 18% ethanol.

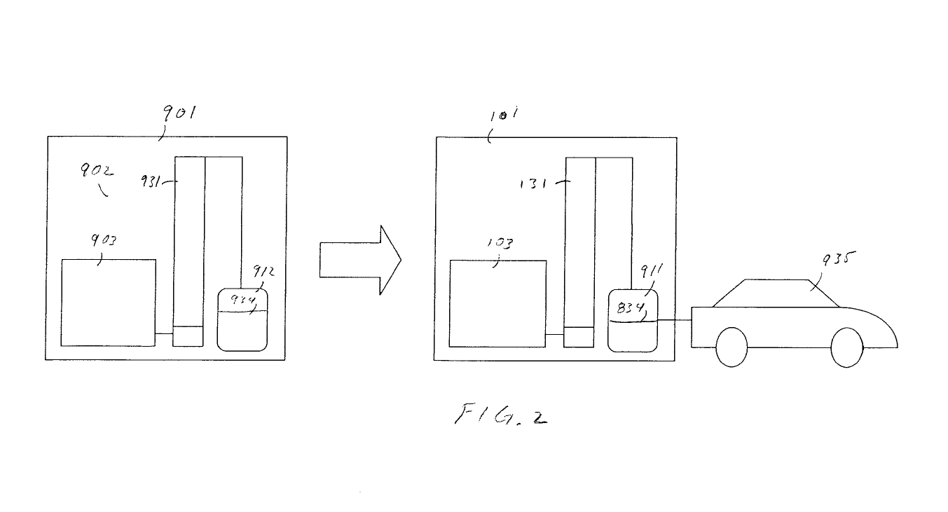

With reference to FIG. 2, in another embodiment, the distributor 901 can have refinery 902 for fermenting and distilling ethanol. The refinery 902 can be a much larger system than the micro-refinery 101 or the refinery 902 can be one or more micro-refineries 101. The feedstock is placed in the fermentation tank 903 and fermented over several days. The fermented liquid can then be distilled through a distillation tube 931 to increase the ethanol content. In an embodiment, the distillation only needs to be between 60 proof to 100 proof ethanol. Thus, high level separation of the water from the ethanol may not be required. The pre-distilled feedstock 934 can be between 30% to 60% ethanol and may be stored in a storage tank 912. When micro-refinery operators order the pre-distilled feedstock 934, it is transported to the micro-refinery 101 where it can be further distilled through the distillation tube 131 and filtration units. The pure ethanol 834 can be stored in a storage tank 911 and used as fuel for internal combustion vehicles 935.

The feedstock, pre-fermented feedstock and pre-distilled feedstock can be transported from the distributor 901 to the micro-refineries 101 in various different ways. In an embodiment, trucks are used to transport the feedstock. The truck can include a storage tank or container for the feedstock. In other embodiments, the feedstock can be transported by any other type of vehicle including: boats, planes, cars, etc. If the distributor 901 and the micro-refineries 101 are in close proximity, the feedstock can be transported by pipelines, conveyor belts or other transportation means.

There are several advantages to the fermentation and partial distillation of the ethanol. The feedstock transportation costs are greatly improved because much more of the transported materials are converted into fuel. Thus, much less pre-fermented or pre-distilled feedstock needs to be transported from the distributor 901 to the micro-refineries 101 to produce the same quantity of ethanol 834. Normally, an operator will purchase feedstock from a distributor 901 and the feedstock must be transported from the distributor to the micro-refinery 101. If unfermented feedstock is purchased, only a small percentage of this material is converted into ethanol 834. The remaining material is mostly waste water that is either recycled or removed through a drain system.

The amount of waste material transported will depend upon the ethanol content of the feedstock. A 30 proof pre-fermented feedstock only contains 15% ethanol. Thus, the remaining 85% is waste water and other feedstock material. Thus, the majority of the transportation costs are used to transport waste material. In contrast, 50% of the 100 proof feedstock can be converted into ethanol. Thus, much more of the transported materials will actually be used as fuel. This can result in a significant transportation savings since much less of the feedstock is water.

While it is possible to distill the feedstock at the distributor 901 up to 200 proof, this may not be desirable. The energy required to distill the feedstock to 200 proof ethanol is not linear. The fermented feedstock may be 30 proof ethanol and additional energy is required to distill the ethanol from 30 proof to 200 proof. The distillation process is initially much more efficient than the final distillation. Approximately one third of the total distillation energy is used to distill the fermented feedstock from 30 proof to 100 proof. However, approximately two thirds of the total distillation energy is required to further distill from 100 proof to 200 proof.

It can be more efficient for a larger refinery system 902 to distill the feedstock to 100 proof than at the micro-refineries 101. However, the distillation from 100 proof to 200 proof may be equally efficient at the large distillation facility 902 and the micro-refineries 101. Thus, ethanol production efficiency is enhanced by this two step distillation process.

Another benefit of the distributor distilling the feedstock to 100 proof rather than 200 proof is improved safety. The 200 proof is highly flammable. In contrast, 100 proof feedstock is half ethanol and half water which makes the 100 proof feedstock much less flammable and much safer to transport. Since many of the micro-refineries 101 can be located in remote areas, the transportation distance can be many miles. In the event of a roadway transportation vehicle accident that results in spillage, 200 proof feedstock can easily start a fire. In contrast, the 100 proof feedstock is unlikely to ignite. Thus, it can be much safer to distribute 100 proof feedstock.

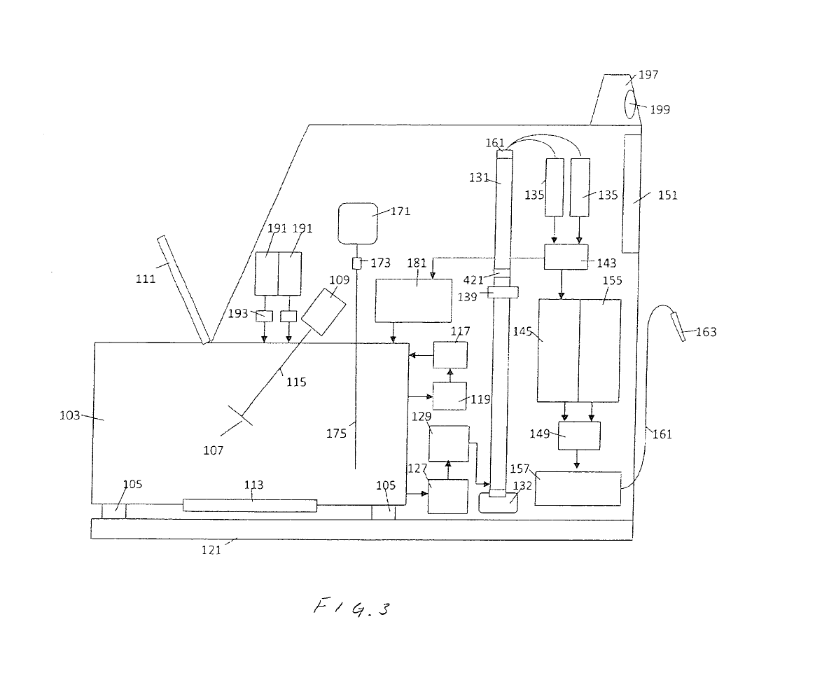

The components of the micro refinery 101 will be described with reference to FIG. 3. The fermentation tank 103 and related components of the micro refinery 101 can be very similar to the fermentation tank 903 of the distributor 901 shown in FIG. 1. However, in an embodiment, the fermentation tank 903 in FIG. 1 is much larger than the fermentation tank 103 in FIG. 3. Similarly, the distillation tube 131 and related components shown in FIG. 3 can be very similar to the distillation tube 931 shown in FIG. 2. However, since the distributor may only be distilling the feedstock to about 100 proof, the distillation process performed by the distributor 901 may not require the ethanol distillation and filtration processes described below with reference to FIG. 3.

In an embodiment the fermentation tank 103 rests on one or more load cells 105 that detect the downward force and produce corresponding electrical output signals. The load cells 105 are coupled to a system controller 151 that monitors the weight of the tank 103 and all contents within the tank 103 throughout the ethanol conversion process. The load cell 105 output signals are proportional to the detected weight. In an embodiment, the system controller 151 can go through a calibration process which detects the weight of the empty tank 103 and stores the empty tank weight as an offset value. The offset value can then be subtracted from any detected weight so that the system controller 151 can detect the weight and quantity of materials that are inserted into the tank 103. The fermentation tank 103 calibration process may be repeated each time a batch of materials is processed.

The system controller 151 may provide a display and/or audio instructions which may indicate the sequence of materials and quantities to be inserted based upon the estimated quantity of ethanol to be produced. For example in an embodiment, a user may input the quantity of ethanol desired. The system then calculates the expected quantities of materials required to produce the desired quantity of ethanol and instructs the user to insert specific quantities of sugar and feedstock. To start the fermentation process, the lid 111 is opened and a specific ratio of sugar and feedstock are inserted into the tank 103.

In an embodiment, the sugar is the first material added to the fermentation tank 103. The weight of the sugar is detected by the system controller 151 and the corresponding volume of water is determined. After the sugar has been added, the system controller 151 can instruct the user to insert the feedstock. The system controller 151 can detect the weight of feedstock and provide instructions and information regarding the quantity of feedstock to add to the fermentation tank. The system controller 151 can detect the weight of the materials being inserted and may provide instructions to the user such as: add more, slow the rate of insertion in preparation to stop and stop. The system controller 151 may have a visual display that indicates the volume of materials added to the tank so the user knows when to stop adding materials to produce the desired volume of ethanol. The system controller 151 may also provide feedback if errors are made. For example, if the system controller 151 detects that too much sugar was added, the system may compensate for this error by increasing the quantity of feedstock to be added to the fermentation tank 103 for the extra sugar.

In another embodiment, the sugar, yeast and other feedstock components such as: phosphorus, sulfur, potassium, magnesium, minerals, amino acids and vitamins can be stored in containers 191 that are coupled to the fermentation tank 103 and the control system 151 can control valves 193 coupled to the containers. Thus, the control system 151 can add the required materials into the fermentation tank 103 so that the insertion of the sugar, yeast and other components is automated. The system may also allow for the large initial quantity of materials to be manually inserted into the fermentation tank and then add additional materials stored in the containers to adjust the batch as necessary. When the proper volume and ratio of feedstock and sugar have been inserted into the fermentation tank 103, the lid 111 is closed. The lid 111 may have a locking mechanism to prevent the addition of any other materials to the tank 103 until after processing is completed.

As discussed, the system controller 151 detects the quantity of sugar in the fermentation tank 103 and calculates the corresponding volume of water for the fermentation process. The system can automatically add the volume of water required for fermentation processing to the tank 103. The proper volume of water can be detected based upon a metered flow of water from a water storage tank 181. Alternatively, the system controller 151 can detect the weight of the water and calculate the volume of water added based upon the known volumetric weight. The system controller 151 is coupled to a valve between the water tank 181 and the fermentation tank 103. The system controller 151 can open the valve to cause water to flow into the tank 103 and when the proper volumetric weight change is detected, the system controller 151 can close the valve. In other embodiments, the water can be added to the fermentation tank 103 manually and the system will indicate when the proper quantity of water has been added.

With the proper mixture of water, feedstock and sugar in the fermentation tank 103 the system can mix the batch ingredients by rotating the agitator 107 to mix the materials. In an embodiment, a motor 109 is used to rotate shaft 115 coupled to an agitating element 107. The agitating element 107 can be an elongated angled mixing blade that circulates liquids in the tank 103 when rotated. The mixing is required to cause the yeast in the feedstock to come in contact with the sugar and nutrients required for fermentation. While a single agitator 107 is illustrated, in other embodiments multiple agitators can be used to mix the materials and prevent clumping of the sugar and feedstock in the corners of the tank 103.

In an embodiment, the control system 151 may detect the proper mixing of the batch materials by the rotational resistance of the agitator 107 or viscosity. A low resistance or viscosity indicates that the agitator 107 is only in contact with water while a higher resistance may indicate that the agitator 107 has contacted a clump of sugar or feedstock. The system can be configured to move the agitator 107 and the shaft 115 within the fermentation tank 103 to completely mix the batch materials. During the mixing process, the rotational resistance is an indication of the status of the mixing. The materials may be properly mixed when the rotational resistance is steady and corresponds to a proper resistance range for the mixture. Once the proper mixed viscosity is detected, the materials are properly mixed and the rotation of the agitator 107 can be stopped or run periodically during the fermentation process.

During the fermentation process, the yeast absorbs the sugar when diluted in water. This reaction produces 50% ethanol and 50% CO₂ by the end of the fermentation process. The chemical equation below summarizes the conversion:

C₆H₁₂O₆(Glucose)=>2CH₃CH₂OH(Ethanol)+2CO₂+heat

In other embodiments, the micro refinery is able to process cellulosic materials to produce ethanol. Cellulosic ethanol is made from plant waste such as wood chips, corn cobs and stalks, wheat straw and sugarcane stalks, stems and leaves or municipal solid plant waste. An advantage for a cellulosic fuel production is that the micro refineries can be configured to process the regional crop plant material, reducing delivery costs. For example, the micro refineries located in the Midwest can be configured to process: wheat straw and corn residue. In the Southern United States the micro refinery can process sugarcane. In the Pacific Northwest and Southeast, wood can be converted into Ethanol.

Corn is easily processed because corn has starches that enzymes can easily break down into sugars and yeast ferments the sugars to produce ethanol. In contrast, cellulosic stalks and leaves contain carbohydrates that are tougher to break down and unravel because they are tightly bound with other compounds. Thus, special processing is required make ethanol from cellulosic farm waste. More specifically, special enzymes are needed in the fermentation tank to break down the carbohydrates. In addition to the special enzymes, the farm waste processing requires genetically engineered bacteria to ferment the farm waste sugars into ethanol.

Another problem with farm waste is that it can be mixed with earth matter such as rocks, clay and gravel that can damage the micro refinery components. In order to prevent damage, the cellulosic materials can be ground with a grinder to more finely chop the materials before processing. The cellulose materials are also separated into glucose and non-glucose sugars using a machine that applies heat, pressure and acid to the cellulosic materials. The heat and pressure produce a sugar and fiber slurry mixture. The non-glucose sugars are washed from the fibers and the glucose based fibers are processed with enzymes to break down and separate the sugars from the fibers. The separated sugars are then fermented with special bacteria microbes into a beer containing ethanol, water and other residue. After fermentation, the micro refinery vaporizes the beer so that the ethanol vapors rise up through a distillation tube to separate the ethanol from water. The vapor from the distillation tube is processed by a porous filter that is used to separate the ethanol vapor from any remaining water vapor as described above.

In another embodiment a different process is used to separate the glucose and non-glucose sugars. The mixture of glucose and non-glucose sugars can be separated, by mixing cellulosic materials with a solution of about 25-90% acid by weight. The acid at least partially breaks down the cellulosic materials and converts the materials into a gel that includes solid material and a liquid portion. The gel is then diluted from about 20% to about 30% by weight and heating the gel, thereby at least partially hydrolyzing the cellulose contained in the materials. The liquid portion can then be separated from the solid material, thereby obtaining a mixed liquid containing sugars and acids. The sugars are then separated from the acids in the mixed liquid by resin separation to produce a mixed sugar liquid containing a total of 15% or more sugar by weight and an acid content of less then 3% by weight.

The method of obtaining the mixed sugar further comprises mixing the separated solid material with a solution of about 25-90% sulfuric acid by weight, thereby further breaks down the solid material to form a second gel that includes a second solid material and a second liquid portion. The second gel liquid is diluted to an acid concentration of from about 20% to about 30% by weight. The diluted second gel liquid is then heated to a temperature between about 80° to 100° C., thereby further hydrolyzing the cellulose remaining in the second gel. The second liquid portion is separated from the second solid material to obtain a second liquid containing sugars and acid. The first and second liquids can be combined to form a mixed liquid. The glucose separation process is described in more detail in U.S. patent application Ser. No. 10/485,285 filed on Jan. 26, 2004, which is hereby incorporated by reference. The described process for producing ethanol from cellulosic materials has many benefits. Tree remains, lawn clippings and other plant debris are normally disposed of in landfill. By using these materials to produce ethanol, the land fill created is significantly reduced, the micro refinery has a substantially free source of feedstock and less greenhouse gases are produced.

A requirement of fermentation is proper temperature control to keep the ingredients within a proper fermentation temperature range. If the yeast temperature is too cold the yeast can become dormant and fermentation is slowed and if the temperature is too high the yeast can be killed. There are various types of yeast, some of which have a high temperature tolerance. The internal temperature of the fermentation tank 103 should be between about 60 and 90 degrees Fahrenheit to preserve yeast culture life. In order to increase the speed of fermentation, the temperature may be maintained at the higher end of the yeast tolerance temperature range.

In an embodiment, the system 101 also includes a thermoelectric mechanism 113 that can be coupled to the fermentation tank 103. The thermoelectric mechanism 113 is powered by a DC electrical power supply and maintains the optimum processing temperature within the tank 103. In order to provide uniform temperature control, a plurality of thermoelectric mechanisms 113 can be attached to various sections of the tank 103. In an embodiment, the system controller 151 is coupled to the thermoelectric mechanism 113 and a temperature transducer is mounted within the fermentation tank 103. The system controller 151 receives a signal corresponding to the internal tank temperature from the temperature transducer and determines if the fermentation tank 103 is within the proper temperature range or if the batch needs to be heated or cooled. As discussed above, the fermentation process produces heat, so in some cases heating or cooling of the tank 103 may not be required. If the system detects that the fermentation tank 103 is too cold, the system controller 151 applies direct current electrical power to the thermoelectric mechanism 113 in the heating mode of operation. If the temperature of the fermentation tank 103 is too hot, the thermoelectric mechanisms 113 can be switch to a cooling mode to reduce the temperature of the tank 103 by reversing the polarity of the electrical power to the thermoelectric mechanism 113. The system controller 151 can also turn the power to the thermoelectric mechanism 113 off when the fermentation tank 103 temperature is within the proper or optimum temperature range for fermentation. The optimum temperature can depend upon the specific type of yeast being fermented but is typically between about 25° C. to 30° C.

In another embodiment, the system may utilize a pump 119 that pumps the batch through a thermoelectric radiator 117 that is separate from the fermentation tank and then returns the batch to the fermentation tank. If the system controller 151 detects that the batch is too cold, the pump 119 is actuated to pump the batch through the thermoelectric radiator 117 which is controlled by the controller 151 to heat the batch. Alternatively, if the system controller 151 detects that the batch is too hot, the pump 119 is actuated to pump the batch through the thermoelectric radiator 117 which is controlled by the controller 151 to cool the batch. The outlet of the thermoelectric radiator 117 can be coupled to the fermentation tank 103 so that all thermally processed batch materials are returned to the fermentation tank 103.

In an embodiment, the system can be used in a wide variety of environments and has the ability to produce ethanol in a wide range of ambient conditions. This requires the cooling of the fermentation tank in hot regions and seasons and heating of the fermentation tank 103 in cold areas and seasons. A larger number of thermoelectric mechanisms 113 can be used in systems located in more extreme ambient temperatures. In an embodiment, the user can simply purchase and install additional thermoelectric mechanisms 113 to compensate for the hotter or colder temperatures. It is also possible to reduce the effects of extreme ambient temperatures by placing the micro refinery system within a protective enclosure and adding insulation to the micro refinery systems.

The thermoelectric mechanisms 113 can be mounted on the fermentation tank 103 walls or, as discussed above with reference to FIG. 3, the thermoelectric mechanisms can be configured as a thermoelectric radiator 117. The fermentation liquid can be pumped through a thermoelectric radiator 117 to provide heating and cooling. Thus, the thermoelectric heating and cooling mechanism 113 and thermoelectric radiator 117 can cool the batch fermentation tank or heat the batch through the system controller 151 by reversing the DC polarity applied to the thermoelectric mechanisms 113 and thermoelectric radiator 117.

In a preferred embodiment, the fermentation tank 103 holds about 200 gallons of liquid. The thermoelectric mechanisms 113 are practical for small fermentation batches in this liquid volume range, but lack enough thermal energy to perform thermal control of larger commercial fermentation processing. For these reasons, the thermoelectric mechanisms can be used with the inventive system to control the temperature of about 200 gallons of liquid but are not suitable for temperature control of a larger 1,000+ gallon commercial fermentation processing tank.

A problem with the fermentation process is that it is not always a predictable process. The time required to complete the fermentation process will vary depending upon the purity of the sugar, and yeast, as well as the batch temperature. One way to monitor the fermentation progress is by monitoring the change in weight of the fermenting liquid. During fermentation, the sugar is converted into ethanol and CO₂ which is vented out of the fermentation tank 103. Thus, the venting of the CO₂ results in a weight reduction of the batch. In an embodiment, the force sensors 105 are used to periodically or continuously check the weight of the batch during the fermentation process. As CO₂ is vented from the fermentation tank 103, the batch gets lighter. The system can monitor the progress of batch fermentation by monitoring changes in the weight of the batch. An initial weight of the batch can be determined and stored in memory. Changes in the batch weight are caused by the conversion of sugar into CO₂ which is vented from the fermentation tank 103. The system controller 151 can determine that the fermentation process is complete when the weight of the batch is reduced by a known percentage. Alternatively, the system controller 151 can determine that the fermentation process is complete when the rate of weight reduction slows or stops. A CO₂ sensor can also be coupled to the fermentation tank. Since the CO₂ is vented, a low level of CO₂ in the tank 103 would indicate that less CO₂ is being produced by the batch.

As discussed above, the force sensors 105 can be used for detecting an initial start weight of the sugar, feedstock and water loaded into the tank 103 at the beginning of the fermentation process. The weight can then be detected periodically by sampling the force sensors 105 at time intervals. By monitoring the weight of the batch over time, the rate of weight change over time can be used to determine the stage of the batch in the fermentation process. For example with reference to FIG. 3, a graphical representation of the weight of the batch over time is illustrated. At the beginning of the process, the weight of the batch drops fairly quickly. As the conversion of the sugar to ethanol progresses, the rate at which the weight decreases slows. Eventually, the weight change becomes very low indicating that the fermentation process is complete.

In addition to detecting the weight of the batch, the system can also perform chemical detection of the batch ingredients. In an embodiment, the micro refinery includes a batch testing mechanism 171 shown in FIG. 3, which can detect the chemical components of the batch and may include an optical, electrical, chemical or any other type of chemical sensor. A delivery mechanism may include a tube 175 that is coupled to a pump 173 to deliver samples of the batch to the testing mechanism 171. The testing mechanism 171 can be coupled to the controller 151 and can be used to check the chemical balance of the batch during the fermentation process. The detected quantity or ratio of batch components from the test mechanism 171 is compared to an optimum value which can be stored on a look up table or provided by another source. The optimum ratio of the batch components can change during fermentation. If there is a significant difference between the measured and optimum values, the controller 151 can transmit a signal indicating the problem and/or the controller 151 may automatically add chemical components to the fermentation tank 103 to rebalance the batch. By continuously testing and adjusting the batch throughout the fermentation process, the ethanol production from the batch can be maximized. More specific examples and descriptions of the sensors used in the chemical testing mechanism are described later.

Although the fermentation tank 103 has been described above for fermenting sugar and feedstock, the inventive system also has the ability to process different materials and can extract ethanol from recycled alcoholic beverages such as beer, wine and other alcohol products. The user can select the function of the micro refinery system as either a sugar fermentation tank or a processor of discarded alcohol. In the sugar fermentation mode, the micro refinery system ferments the sugar to create alcohol as described above. In the alcohol recycling mode, the alcoholic products also go into the fermentation tank prior to being processed by a distillation system for conversion into ethanol. The multi-function design provides a market advantage for recycling either sugar or discarded alcohol commonly found at bar restaurants or wineries.

After or during the fermentation of the sugar, it is possible to add the alcoholic liquids to the fermentation tank. The processor can indicate when alcoholic beverages can be added. In an embodiment, the controller can actuate a locking mechanism coupled to the lid 111 to allow or prevent the user from adding materials to the fermentation tank 103. Because the reaction of the yeast has converted much of the liquid into carbon dioxide, the volume of liquids in the fermentation tank 103 will decrease after fermentation is complete which allows room for recycling the alcoholic beverages. The micro refinery will then separate the ethanol from the batch as well as the alcohol from the discarded beverages and the other liquid components.

The ethanol is separated from the water and other liquids by processing the fluids through a distillation system. In an embodiment, the distillation system of the present invention includes a pump 127, a heater 129, a distillation tube 131 and a gimbaled mechanism 139 that is used to position the distillation tube 131 in a vertical orientation.

The vertical orientation can be maintained by a gyroscope 132 mounted to the distillation tube 131. The gyroscope 132 includes a rotor that can be aligned with the vertical axis of the distillation tube and a motor that rotates the rotor. The rotation of the rotor stabilizes the gyroscope 132 and distillation tube from any rotational movement. The control system 151 controls the pump 127 to pump the liquids in the fermentation tank 103 through the heater 129 to cause the water and ethanol to boil and vaporize. As discussed above, heat can be transferred to the heater 129 through a heat exchange loop to improve the efficiency. The vaporized liquid is directed to the bottom of the distillation tube 131. As the vapors travel higher through the distillation tube 131, the ethanol molecules separate from the water molecules and exit the upper part of the column. If water and other non-ethanol liquids vaporize, these vapors will tend to be condensed on the sides of the distillation tube as they cool in the distillation tube 131. The condensed liquids may then adhere or drip down the inner walls of the distillation tube 131 rather than exiting the top of the tube 131. The distillation system may also include one or more temperature sensors which monitor the vapor temperature and control the heater 128 to produce vapor at an optimum separation temperature. Excessive heat will cause a faster vapor velocity resulting in more water exiting the distillation tube 131, while a low temperature vapor temperature will result in a low flow of ethanol from the distillation tube 131.

The distillation process requires that the distillation tube 131 be in a perfect vertical alignment. The vapors slowly rise vertically straight up and the flow path is preferably undisturbed by sidewalls as the vapors travel up through the center of the distillation tube 131 and out from the top. If the distillation tube 131 is out of alignment, the rising vapors will run into the side of the tube 131 resulting in condensation of ethanol vapors and reducing the efficiency of the distillation system. Similarly, water vapor rising on the side wall tilted away from vertical may not condense on the sidewalls reducing the separation of the water and ethanol. Thus, perfect vertical alignment is necessary for the high efficiency distillation.

In an embodiment, a gyroscope 132 shown in FIG. 3 is mounted to the bottom of the distillation tube 131. The gyroscope 132 includes a rotor and a motor that rotates the rotor. Because the weight of the gyroscope 132 is supported by the distillation tube 131, the center of gravity of the gyroscope 132 can be aligned with the vertical center axis of the distillation tube 131 so the weight will not cause misalignment. The rotational axis of the rotor can be aligned with the vertical axis of the distillation tube and while the rotor is rotating the gyroscope 132 and distillation tube 131 are stabilizes so that any angular motion of the micro refinery will not alter the vertical alignment of the distillation tube. In an embodiment, a distillation tube 131 is vertically aligned before the gyroscope is turned on and the rotor starts spinning.

The distillation tube 131 can be fragile and in some cases it may be desirable to lock the distillation tube 131 in place to prevent movement. In an embodiment, the vertical alignment system includes a locking mechanism that prevents the distillation tube from rotating. In an embodiment, the system can detect ambient conditions through sensors such as wind meters and/or accelerometers coupled to the housing. If the wind speed is very high, the system may move which will cause the distillation tube to move out of vertical alignment. Rather than risking damage to the distillation tube, the system may have a “safe” mode that can be actuated when predetermined wind speed or acceleration movement is detected. For example, the micro refinery may go into a safe mode with the distillation tube and other fragile system components locked in a safe position, when the detected winds are greater than 40 MPH are detected or an earthquake greater than 5.0 is detected. The system may also receive weather warnings for its geographic location from an outside source such as the internet weather information services and respond to storm warnings by scheduling safe mode times. The controller may also shut off power and/or provide surge protection to prevent damage to the electrical components due to power surges or power outages.

In an embodiment, the distillation tube can be filled with material packing or horizontal perforated plates which are used to strip vaporized beer from the alcohol. Ideally, the vaporized beer and ethanol enter the bottom of the distillation tube and the combined vapor travels up the tube. Water and other heavier material are blocked by packing or plates. In contrast, the ethanol will tend to stay in vapor form and continue to travel up the distillation tube. This helps to separate the water and other contaminants from the ethanol vapor. The plates can be horizontally oriented within the tube and multiple plates can be positioned along the length of the distillation tube. A potential problem occurs when the micro refinery temporarily stops production. The water will condense or evaporate and the beer can remain on the packing or perforated plates causing clogging of the perforations or packing when the system is used again. The entire condensation tube may need to be cleaned before the system can be used again.

During the normal operation of the micro refinery, the hot ethanol and water vapors exit the distillation tube 131 and travel through a membrane system 135 which separates water molecules from the ethanol molecules. The membrane system 135 includes a porous separation membrane that can be made of ceramic, glass or very course materials. As discussed above, the filtration system is used to further separate ethanol from water. However, if 100 proof feedstock is being produced, this higher filtration may not be necessary. Thus, the refinery 902 illustrated in FIG. 2 may not have the filters 135 or the gasoline blending features of the micro-refinery illustrated in FIG. 3.

A potential problem with the porous membrane system is that the membrane materials can be susceptible to this thermal damage. In particular, “thermal damage” of the membrane can occur if the temperature of the ethanol vapor is substantially hotter than the membrane. For example, the membrane may be at ambient temperature and then immediately exposed to hot ethanol vapor resulting in damage. To prevent thermal damage of the membrane a micro controlled warming system is used to pre-heat the membrane to ensure the membrane temperature is suitable for processing the hot vapor. In an embodiment, the temperature of the membrane is detected by a thermocouple attached to the membrane system. As the control system directs the flow of fluids out of the fermentation tank through to the heater and distillation tube, it detects the temperature of the membrane before the hot vapors are directed to the distillation tube. With reference to FIG. 3, if the membrane is cold, the system controller 151 can activate a heating element and monitor the membrane temperature. As the membrane temperature increases, the control system may have a thermostatic setting to prevent over heating of the membrane by the heater. When the membrane temperature is pre-heated to a safe temperature, the system controller 151 can allow hot vapors to flow through the distillation tube 131 to the membrane. Once the hot vapors are flowing through the membrane, the vapors will heat the membrane and power to the heating element can be removed. In order to assist with the ethanol and water separation process, the water vapor can be drawn through the porous membrane with a vacuum 143.

In an embodiment, the membrane system 135 can have a back up membrane 135. If one membrane system 135 is damaged, the controller will detect the failure and the controller 151 can actuate a valve 136 to divert the water and ethanol vapors from the distillation tube 131 to the back up membrane system 135. The controller 151 can transmit a signal indicating that the membrane 135 is damaged through the transceiver 197 to an operator or maintenance group. The damaged membrane system 135 can then be replaced while the water and ethanol vapors are separated by the backup membrane system 135.

After passing through the membrane system 135 and vacuum 143, the water can condense and flow into the water storage tank 181 before being used again in the fermentation tank 131. The separated ethanol exits the membrane system 135 and then flows through a thermo-electric cooler 166 which causes the ethanol to condense into a liquid. The liquid ethanol then flows into a storage tank 145 where it is stored before being mixed with gasoline. An ultrasonic or other liquid sensor coupled to the storage tank 145 can detect the liquid ethanol level within the storage tank 145 and provide this ethanol production information to the system controller 151. In an embodiment, the system controller 151 can detect when the ethanol storage tank 145 is full and stop the distillation process until there is available space in the storage tank 145.

In an embodiment, the inventive micro refinery can mix the ethanol stored in the ethanol storage tank 145 with gasoline that is stored in a gasoline storage tank 155 in any ratio set by the user through the system controller 151. The control system includes a user interface which allows the user to select the desired fuel blend ratio. The system may include a lock that prevents the fuel mixture setting to exceed the maximum or minimum allowable ethanol percentage for the vehicle. Once the fuel mixture has been selected, the user can use the micro refinery functions like a normal gasoline pump. The user removes the nozzle 163 from a cradle on the micro refinery 101 and places it in the tank filler of the vehicle. A lever coupled to the nozzle 163 is actuated to start the pumps 149 which cause the fuel to flow from the tanks 145 and 155 through the hose reel 157, the hose 161 and nozzle 163 to the tank of the vehicle. The system will run the ethanol and gasoline pumps 149 at different flow rates to produce the specified fuel ratio. The nozzle 163 will detect when the vehicle tank is full and automatically stop the flow of fuel through the nozzle 163. When the vehicle tank is full, the user places the nozzle 163 back in the cradle and replaces the cap on the fuel filler to end the filling process. With the ethanol tank 145 at least partially drained, the system can begin to produce more ethanol.

The mix ratio of ethanol and gasoline or other fuels can depend upon the type of vehicle being fueled. The use of pure ethanol in internal combustion engines is only possible if the engine is designed or modified for that purpose. However, ethanol can be mixed with gasoline in various ratios for use in unmodified automobile engines. In the United States, normal cars designed to run on gasoline may only be able to use a blended fuel containing up to 15% ethanol. In contrast, U.S. flexible fuel vehicles can use blends that have less than 20% ethanol or up to 85%. The ethanol fuel blend is typically indicated by the letter “E” followed by the percentage of ethanol. For example, typical ethanol fuel names include: E5, E7, E10, E15, E20, E85, E95 and E100, where E5 is 5% ethanol and 95% gasoline, etc.

After the processing performed by each of the micro refinery systems is complete, the micro refinery systems may also be cleaned. In an embodiment, the micro refinery includes cleaning mechanisms that can spray the fermentation tank with pressurized soap and water which will remove particulates from the tanks and other components. The system can then rinse the system components to remove the soap and other residue. In an embodiment a drain valve is opened to allow the waste liquids from the fermentation tank and the distillation system to drain from the system through a drain hose. The system may include an automated cleaning system that utilizes valves coupled between a water supply and a spray nozzle that emits high pressure water and is actuated by the system controller. The spray can be directed towards the fermentation chamber walls to remote deposited materials. As the volatile materials have been removed from the interior surfaces of the micro refinery, a drain valve is opened and the waste materials can be poured down into public drainage systems.

Because the micro refinery is a complex mechanism, sensors and controls are used to automate the operation and optimize the ethanol production performance. The micro refinery can include various sensors that monitor the operating conditions of the processing systems including: the fermentation tank, the load cell weight detection system, the temperature control system, the mixing agitator for the fermentation tank, the distillation system, the membrane separation system, the storage tank and a blending and pumping system. All of these systems include sensors that are coupled to the controller.

It will be understood that the inventive system has been described with reference to particular embodiments, however additions, deletions and changes could be made to these embodiments without departing from the scope of the inventive system. For example, the same processes described can also be applied to other devices. Although the systems that have been described include various components, it is well understood that these components and the described configuration can be modified and rearranged in various other configurations.

What is claimed is:

1. A ethanol production system comprising:
a fermentation unit having a fermentation tank for producing a feedstock having 12% to 18% ethanol; and
a distillation unit for distilling the feedstock to produce feedstock having 40% to 60% ethanol; and
a transportation unit for delivering the feedstock having 40% to 60% ethanol.
